(19) United States
(12) Patent Application Publication
(10) Pub. No.: US 2024/0209303 A1
(43) Pub. Date: Jun. 27, 2024

(54) CULTURE MONITORING SYSTEM AND METHODS

(71) Applicant: Baebies, Inc., Durham, NC (US)

(72) Inventors: Rainer Ng, Durham, NC (US); Vamsee Pamula, Durham, NC (US)

(73) Assignee: Baebies, Inc., Durham, NC (US)

(21) Appl. No.: 18/287,954

(22) PCT Filed: Apr. 22, 2022
(86) PCT No.: PCT/US2022/025952

§ 371 Date: Oct. 23, 2023

Related U.S. Application Data
(60) Provisional application No. 63/178,138, filed on Apr. 22, 2021.
(60) Provisional application No. 63/226,287, filed on Jul. 28, 2021.
(60) Provisional application No. 63/243,875, filed on Sep. 14, 2021.

(51) Int. Cl. C12M 1/00; C12M 3/06; C12Q 1/18; C12Q 1/686

(52) U.S. Cl. CPC C12M 41/14; C12M 23/16; C12M 29/00; C12Q 1/18; C12Q 1/686

(57) ABSTRACT

An integrated culture monitoring system and methods are provided. The system may include an incubation chamber, a temperature control device arranged to control temperature of the incubation chamber, a fluid-handling cartridge (e.g., a digital microfluidics (DMF) cartridge), and a fluid passage coupled to a pump and providing a fluid passage between the incubation chamber and the fluid-handling cartridge. The system may include a processor programmed to control the temperature control device to increase or decrease the temperature, and a pump to control movement of fluid through the fluid passage and dispensing and assaying of a droplet of culture fluid. Also provided are methods of using the system for screening for sepsis.

### RELATED APPLICATIONS

The presently-disclosed subject matter is related and claims priority to U.S. Provisional Patent Application No. U.S./63/178,138, entitled “INTEGRATED CULTURE MONITORING SYSTEM AND METHOD,” filed on Apr. 22, 2021; U.S. Provisional Patent Application No. U.S./63/226,287, entitled “CULTURE MONITORING SYSTEM AND METHOD,” filed on Jul. 28, 2021; and U.S. Provisional Patent Application No. U.S./63/243,875, entitled “CULTURE MONITORING SYSTEM AND METHOD,” filed on Sep. 14, 2021; the entire disclosures of which are incorporated by reference.

### TECHNICAL FIELD OF THE INVENTION

The presently disclosed subject matter relates generally to methods of screening for sepsis and more particularly to an integrated culture monitoring system.

### BACKGROUND

Sepsis is a life-threatening condition that occurs when the body's response to infection causes injury to its own tissues and organs. Sepsis develops when a pathogen is released into the bloodstream and causes inflammation throughout the entire body. In the early stages, it is difficult to differentiate sepsis from other diseases because certain symptoms of sepsis, such as fever, increased heart rate, and breathing rate, mimic the symptoms of other diseases. The ability to detect sepsis at its earliest stages is critical because early sepsis is usually reversible with antibiotics, fluids, and other supportive medical interventions. However, as time progresses the risk of mortality increases substantially. Therefore, early detection of sepsis is desirable.

Currently, blood culture is the gold standard for the detection of microbial pathogens in the bloodstream. This method, however, possesses some intrinsic limitations, as it is laborious and can only identify microbes that grow under optimized culture conditions. Also, this method is also difficult in establishing the diagnosis for patients who are already taking antibiotics. Molecular diagnostics is an emerging technology that is increasingly used to provide specific pathogenic diagnoses after culturing. However, molecular diagnostics is performed discretely after culturing requiring manual intervention.

Therefore, new approaches are needed with respect to providing rapid diagnostic procedures to complement, improve, and/or replace current sample culture approaches.

### SUMMARY OF THE INVENTION

The invention provides an integrated culture monitoring system.

In one embodiment, the integrated culture monitoring system may comprise: (a) an incubation chamber; (b) a temperature control device arranged to control temperature of the incubation chamber; (c) a fluid-handling cartridge; (d) a fluid passage operationally coupled to a pump and providing a fluid passage between: (i) the incubation chamber; and (ii) the fluid-handling cartridge; and arranged to permit the flow of fluid between the incubation chamber and the fluid-handling cartridge.

In another embodiment, the integrated culture monitoring system may further comprise one or more processors: (a) electronically coupled to the temperature control device, the fluid-handing cartridge, and the pump; and (b) programmed to control one or more of: (i) the temperature control device to increase or decrease temperature; (ii) the pump to control movement of fluid through the fluid passage; and/or (iii) dispensing and assaying of a droplet of culture fluid pumped via the fluid passage into the fluid-handling cartridge.

In yet another embodiment, the fluid-handling cartridge may be a digital microfluidics (DMF) cartridge.

In still another embodiment, the incubation chamber may include culture fluid.

In another embodiment, the culture fluid may be selected from a group consisting of whole blood, blood fractions, urine, other bodily fluids, culture media, or combinations of the foregoing.

In yet another embodiment, the fluid-handling cartridge may comprise reagents for performing one or more assays on a sample of the culture fluid.

In still another embodiment, the system may further comprise one or more sensors electronically connected to and controlled by one or more system processors.

In another embodiment, the one or more sensors may be arranged to detect a reaction in a droplet on the fluid-handling cartridge.

In yet another embodiment, the incubation chamber may be fluidly connected to the fluid-handling cartridge.

In still another embodiment, the integrated culture monitoring system may further comprise an agitation mechanism mechanically coupled to or otherwise contacting the incubation chamber.

In another embodiment, the one or more sensors may comprise an optical sensor arranged to detect bacteria in culture fluid.

In yet another embodiment, the optical sensor may be selected from a group consisting of a fluorimeter, a charge-coupled device, a photodetector, a spectrometer, a photodiode array, or any combination thereof.

In still another embodiment, the fluid-handling cartridge may comprise two substrates separated by a gap that forms a chamber in which droplet operations are performed.

In another embodiment, the incubation chamber may comprise a culture bottle.

In yet another embodiment, the integrated culture monitoring system may further comprise an output device displaying a graphical user interface (GUI) electronically coupled to the processor, and the processor is programmed to provide assay results to a system user.

In still another embodiment, the one or more assays may comprise a PCR assay for detecting the presence of bacteria.

In another embodiment, the PCR assay may comprise broad-range 16S PCR.

The present invention provides a method for conducting an assay or assays in a device.

In one embodiment, the method may comprise the steps of: (a) providing the integrated culture monitoring system of any of the preceding claims; (b) providing a culture fluid in the incubation chamber; (c) using the processor causing: (i) the temperature control device to control temperature in the incubation chamber; (ii) the pump to pump a sample of culture fluid from the incubation chamber into the fluid-handling cartridge; and/or (iii) the fluid-handling cartridge to conduct one or more assays on a sample of the culture fluid.

In another embodiment, the processor may cause the pump to pump a sample of culture fluid from the incubation camber into the fluid-handling cartridge and the one or more assays may be performed on the sample at a regular time interval.

In yet another embodiment, the regular time interval may be from about every two minutes to about every thirty minutes.

In still another embodiment, the one or more assays may comprise an assay for identifying a bacterial species.

In another embodiment, the one or more assays may comprise an assay for performing antimicrobial susceptibility testing.

In yet another embodiment, the one or more assays may comprise two assays, an assay for identifying bacterial species and an assay for performing antimicrobial susceptibility testing in the same device.

In still another embodiment, the identification of a bacterial species and antimicrobial susceptibility testing may both be completed within about eight hours, within about six hours, within about four hours, within about two hours, or within about one hour.

In another embodiment, the one or more assays may comprise a PCR assay to detect the presence or absence of bacteria in the sample and/or may comprise a PCR assay for antimicrobial susceptibility testing.

The present invention provides a method for detecting and characterizing bacteria in a sample.

In one embodiment, the method may comprise the steps of: (a) providing an integrated culture monitoring system; (b) obtaining a first sample from the incubation chamber and transporting the first sample through a fluid passage to a fluid-handling cartridge; (c) performing sample preparation steps on the cartridge using droplet operations; and (d) performing one or more assays on the prepared first sample to detect the presence or absence of bacteria and/or to characterize bacteria in the first sample on the fluid-handling cartridge.

In another embodiment, the fluid-handling cartridge may be a digital microfluidics (DMF) cartridge.

In still another embodiment, the integrated culture monitoring system may comprise the integrated culture monitoring system of the present invention.

In yet another embodiment, the first sample of culture fluid may be obtained from the incubation camber and may be transported into the fluid-handling cartridge and the one or more assays may be performed on the sample at a regular time interval.

In another embodiment, the regular time interval may be from about every two minutes to about every thirty minutes.

In still another embodiment, the one or more assays may comprise an assay for identifying a bacterial species.

In yet another embodiment, the method may further comprise the following steps if bacteria is detected in step (d): (e) obtaining a second sample from the incubation chamber and transporting the second sample through the fluid passage to the fluid-handling cartridge; (f) performing sample preparation steps on the fluid-handling cartridge using droplet operations; and (g) performing an assay on the prepared second sample to determine bacterial species and to inform antibiotic susceptibility in the second sample on the fluid-handling cartridge.

In still another embodiment, the identification of a bacterial species and antimicrobial susceptibility testing may both be completed within about eight hours, within about six hours, within about four hours, within about two hours, or within about one hour.

In yet another embodiment, the one or more assays may comprise a PCR assay to detect the presence or absence of bacteria in the sample and/or may comprise a PCR assay for antimicrobial susceptibility testing.

The present invention provides a method for detecting the presence or absence of bacteria in a sample.

In one embodiment, the method may comprise the steps of: (a) providing an integrated culture monitoring system, wherein the integrated monitoring system comprises an incubation chamber for holding a sample, a digital microfluidics (DMF) cartridge, a processor and a graphical user interface (GUI); (b) providing a fluid connection between the incubation chamber and the DMF cartridge; (c) causing a processor to: (i) activate the integrated culture monitoring system to flush a liquid interface between the incubation chamber and the DMF cartridge; and/or (ii) initiate a pump to pump a first sample or a first portion of the first sample from the incubation chamber into the DMF cartridge; (d) performing an assay to detect the presence or absence of bacteria on the cartridge; (e) processing the assay information using the processor; and (f) reporting the assay results via the GUI of the system.

In another embodiment, step (d) may comprise performing broad-range 16S PCR on the first pumped sample or the first portion of the first pumped sample to detect the presence or absence of bacteria on the DMF cartridge.

In yet another embodiment, the method may further comprise the step of transporting the first sample or the first portion of the first sample to a waste receptacle.

In still another embodiment, the method may further comprise the following steps if bacteria are detected in the first pumped sample or in the first portion of the first pumped sample by an assay: (a) using the processor to: (i) activate the integrated culture monitoring system of any of the preceding claims to flush the liquid interface between the incubation chamber and the DMF cartridge; and (ii) initiate the pump to obtain a second sample or a second portion of the first sample and transport the sample through the fluid passage to the DMF cartridge; (b) performing sample preparation on the second pumped sample or the second portion of the first pumped sample on the DMF cartridge; (c) performing a molecular identification (ID) test on the bacteria to determine a bacterial species; (d) processing the test information using the processor; and (e) reporting the test results via the GUI of the system.

In another embodiment, if the presence of bacteria is detected, then the method may further comprise: using the processor to: activate the integrated culture monitoring system of any of the preceding claims to flush the liquid interface between the incubation chamber and the DMF cartridge.

In yet another embodiment, the method may further comprise using the processor to cause the pump to pump a third sample, a second portion of the second sample, or a third portion of the first sample from the incubation chamber into the DMF cartridge.

In still another embodiment, the processor may cause the pump to pump a third sample, a second portion of the second sample, or a third portion of the first sample from the incubation chamber into the DMF cartridge at a regular time interval.

In another embodiment, the regular time interval may be from about every two minutes to about every thirty minutes.

In yet another embodiment, the method may further comprise using the processor to cause the DMF cartridge to perform sample preparation on the third sample, on a second portion of the second sample, or on the third portion of the first sample.

In still another embodiment, the method may further comprise using the processor to cause the DMF cartridge to perform molecular and/or phenotypic antibiotic susceptibility testing (AST) on the third prepared sample, second portion of the second prepared sample, or the third portion of the first prepared sample to determine the presence or absence of antibiotic susceptibility gene markers.

In another embodiment, the method may further comprise determining Gram status of the detected bacteria.

In yet another embodiment, the method may further comprise using the processor to analyze the assay, the ID test, and/or the AST test results.

In still another embodiment, the method may further comprise using the processor to report the assay, the ID test, and/or the AST test results via the GUI of the system.

In another embodiment, the DMF cartridge may perform host response testing on a sample.

In yet another embodiment, the method may further comprise performing a molecular ID test, molecular AST test and/or phenotypic AST test, and Gram status test concurrently on the third prepared sample, the second portion of the second prepared sample, or on the third portion of the first prepared sample.

In still another embodiment, the molecular ID test may detect bacteria from a group consisting of E. coli, S. agalactiae, S. aureus, S. epidermidis, and Klebsiella pneumoniae.

In another embodiment, the molecular AST test may detect antibiotic gene markers from a group consisting of mec A/C, aminoglycosides, and carbapenem-resistant Enterobacteriaceae.

In yet another embodiment, the Gram status test may detect the presence of Gram-positive and/or Gram-negative bacteria.

In still another embodiment, the detection of bacteria, the Gram status test, ID test, and/or the AST test may be completed within about eight hours, within about six hours, within about four hours, within about two hours, or within about one hour.

In another embodiment, the incubation chamber may be a liquid receptacle.

In yet another embodiment, the liquid receptacle may be a culture bottle.

In still another embodiment, the molecular ID test, molecular AST test, phenotypic AST test, and/or Gram status test may comprise performing PCR-based testing.

In another embodiment, the reagents may be selected from a group consisting of sample purification reagents, sample lysis reagents, and/or sample concentration reagents.

In yet another embodiment, the reagents may be selected from a group consisting of reagents required to perform broad-range 16S PCR, molecular ID PCR, molecular AST PCR, phenotypic AST PCR, and/or Gram status PCR.

In still another embodiment, the reagents may comprise dried reagents that are reconstituted prior to use.

In another embodiment, the fluid-handling cartridge may be disposable.

In yet another embodiment, the sample may be selected from a group consisting of a subject's blood sample, blood fraction, or other physiologically-derived samples such as swabs, urine, or cerebrospinal fluid.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### BRIEF DESCRIPTION OF THE DRAWINGS

The features and advantages of the present invention will be more clearly understood from the following description taken in conjunction with the accompanying drawings, which are not necessarily drawn to scale, and wherein:

FIG. 1 illustrates a block diagram of an example of a culture monitoring system, which is an example of the presently disclosed integrated culture monitoring system including an instrument and/or device;

FIG. 2 illustrates a block diagram of an example of a cartridge (or device) of the culture monitoring system shown in FIG. 1;

FIG. 3 illustrates a perspective view of an example of the cartridge (or device) and culture bottle of the culture monitoring system shown in FIG. 1;

FIG. 4 illustrates a perspective view of an example of an instrument of the culture monitoring system shown in FIG. 1;

FIG. 5 and FIG. 6 illustrate a flow diagram and a pictorial view, respectively, of an example of a method of using the culture monitoring system shown in FIG. 1 according to the simplest configuration, which is an example of a method of using the presently disclosed integrated culture monitoring system including an instrument and/or device;

FIG. 7A and FIG. 7B illustrate a flow diagram of an example of a method of using the culture monitoring system shown in FIG. 1, which is another example of a method of using the presently disclosed integrated culture monitoring system including an instrument and/or device;

FIG. 8 illustrates a block diagram of another example of a cartridge (or device) of the culture monitoring system shown in FIG. 1 that further supports phenotypic AST PCR assay processes;

FIG. 9 illustrates a flow diagram of an example of a method of using the culture monitoring system shown in FIG. 8 that includes phenotypic AST processes, which is another example of a method of using the presently disclosed integrated culture monitoring system including an instrument and/or device;

FIG. 10 illustrates a block diagram of an example of a culture monitoring system that further supports host response PCR processes, which is another example of the presently disclosed sepsis detection system including an instrument and/or device;

FIG. 11 illustrates a block diagram of an example of a cartridge (or device) of the DMF sepsis detection system shown in FIG. 10; and

FIG. 12 illustrates a flow diagram of an example of a method of using the culture monitoring system shown in FIG. 10 that includes host response PCR processes, which is another example of a method of using the presently disclosed sepsis detection system including an instrument and/or device.

### DETAILED DESCRIPTION OF THE INVENTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides an integrated culture monitoring system and method including an instrument and/or device. For example, an integrated culture monitoring system and method may provide an integrated sepsis diagnostic system including microfluidics-based blood culture and detection mechanisms.

In some embodiments, the presently disclosed integrated culture monitoring system and method may include an instrument for receiving a standard culture bottle (e.g., a blood culture bottle) and a (fluid-handling) cartridge (or device) and wherein the culture bottle may be fluidly coupled to the (fluid-handling) cartridge (or device).

In some embodiments, the presently disclosed integrated culture monitoring system and method may be used to substantially continuously monitor a standard culture bottle using DMF-based technology, such as an instrument and/or a cartridge (or device).

In some embodiments, the presently disclosed integrated culture monitoring system and method may provide one or more sample preparation processes, one or more broad-range 16S polymerase chain reaction (PCR) processes, one or more molecular PCR (and/or phenotypic PCR) processes, a PCR results algorithm, and wherein these processes may be DMF-based processes.

In some embodiments, the presently disclosed integrated culture monitoring system and method provide molecular PCR processes that may include, for example, a molecular identification (ID) PCR process, a molecular antibiotic susceptibility testing (AST) PCR process and/or a phenotypic AST PCR process, and a PCR process for determining whether bacteria are gram-positive or gram-negative, and wherein these processes may be DMF-based processes.

In some embodiments, the presently disclosed integrated culture monitoring system and method may provide one or more sample preparation processes, one or more broad-range 16S PCR processes, one or more molecular PCR processes, a PCR results algorithm, as well as one or more phenotypic AST processes, and wherein these processes may be DMF-based processes.

In some embodiments, the presently disclosed integrated culture monitoring system and method including an instrument and/or device may include a cartridge (or device) configured for processing a sample, e.g., blood, etc., for determining a host response signature to delineate between a bacterial and a non-bacterial (e.g., viral) infection, wherein these processes may be DMF-based processes.

### Culture Monitoring System

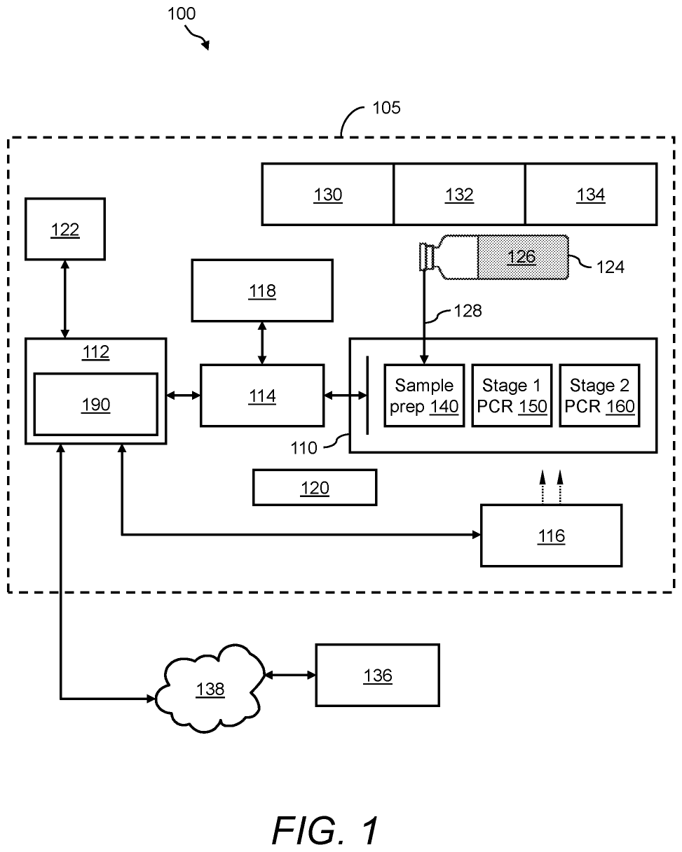

Referring now to FIG. 1 is a block diagram of an example of a culture monitoring system 100, which is an example of the presently disclosed integrated culture monitoring system including an instrument and/or device. In this example, culture monitoring system 100 may include a (fluid-handling) cartridge 110. Cartridge 110 may be, for example, any device or cartridge, droplet actuator, and the like that may be used to facilitate DMF capabilities generally for fluidic actuation. Cartridge 110 of culture monitoring system 100 may be provided, for example, as a disposable and/or reusable device or cartridge.

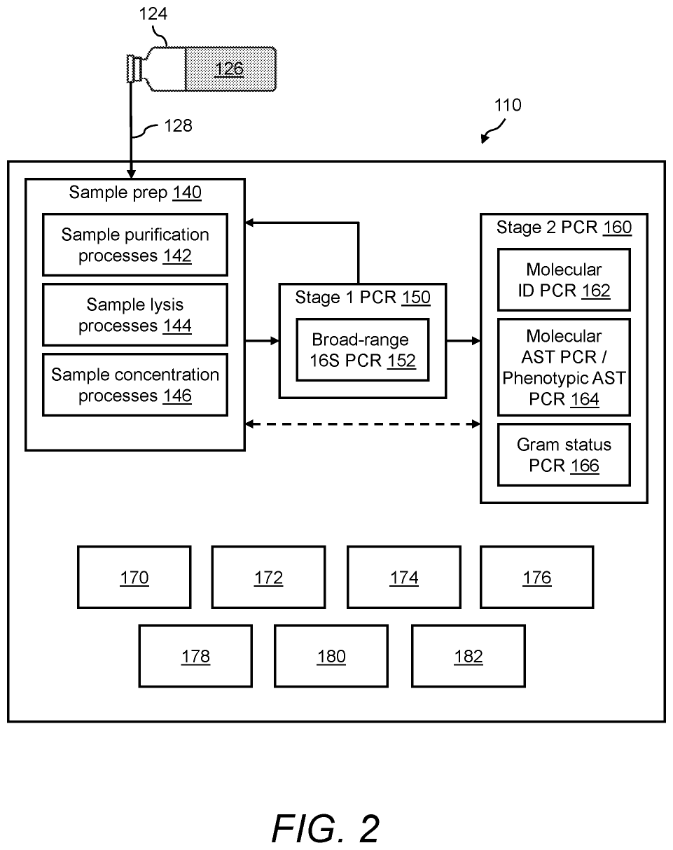

DMF capabilities may generally include but are not limited to, transporting, merging, mixing, splitting, dispensing, diluting, agitating, deforming (shaping), and other types of droplet operations. Applications of these DMF capabilities may include, for example, sample preparation, waste removal, PCR operations, and the like. Generally, culture monitoring system 100 and cartridge 110 may be used to process biological materials. However, particular to culture monitoring system 100, in one example, cartridge 110 possesses DMF capabilities that may be used to support an integrated sepsis diagnostic system including microfluidics-based blood culture and detection mechanisms. For example, such DMF capabilities of cartridge 110 may be used to substantially continuously monitor a standard blood culture bottle using DMF-based technology, such as instrument 105 and/or cartridge 110. More details of cartridge 110 are described hereinbelow with reference to FIG. 2.

Culture monitoring system 100 may further include a controller 112, an interface 114, a detection system 116, certain thermal control electronics 118, one or more magnets 120, and a graphical user interface (GUI) 122. Controller 112 may be electrically coupled to the various hardware components of culture monitoring system 100, such as cartridge 110, detection system 116, thermal control electronics 118, magnets 120, and GUI 122. In particular, controller 112 may be electrically coupled to cartridge 110 via interface 114, wherein interface 114 may be, for example, a pluggable interface for connecting mechanically and electrically to cartridge 110.

Detection system 116 may be any detection mechanism that can be used to accurately determine the presence or absence of a defined analyte and/or target component in different materials and to sensitively quantify the amount of analyte and/or target components present in a sample. Detection system 116 may be, for example, an optical measurement system that may include one or more illumination sources (not shown) and one or more optical measurement devices (not shown). For example, detection system 116 may be a fluorimeter that provides both excitation and detection. In this example, an illumination source and an optical measurement device may be arranged with respect to cartridge 110. An illumination source may be, for example, a light source for the near-visible range (360-800 nm), such as but not limited to, a white light-emitting diode (LED), a halogen bulb, an arc lamp, an incandescent lamp, lasers, and the like. An optical measurement device may be used to obtain light intensity readings. The optical measurement device may be, for example, a charge-coupled device, a photomultiplier tube, a photodetector, a spectrometer, a photodiode array, or any combinations thereof.

Most chemical and biological processes require precise and stable temperature control for optimal efficiency and performance. Accordingly, thermal control electronics 118 may be any mechanism for controlling the operating temperature of cartridge 110. Thermal control electronics 118 may include, for example, any thermal sensors for controlling heaters (e.g., Peltier elements and resistive heaters) and/or coolers arranged with respect to cartridge 110.

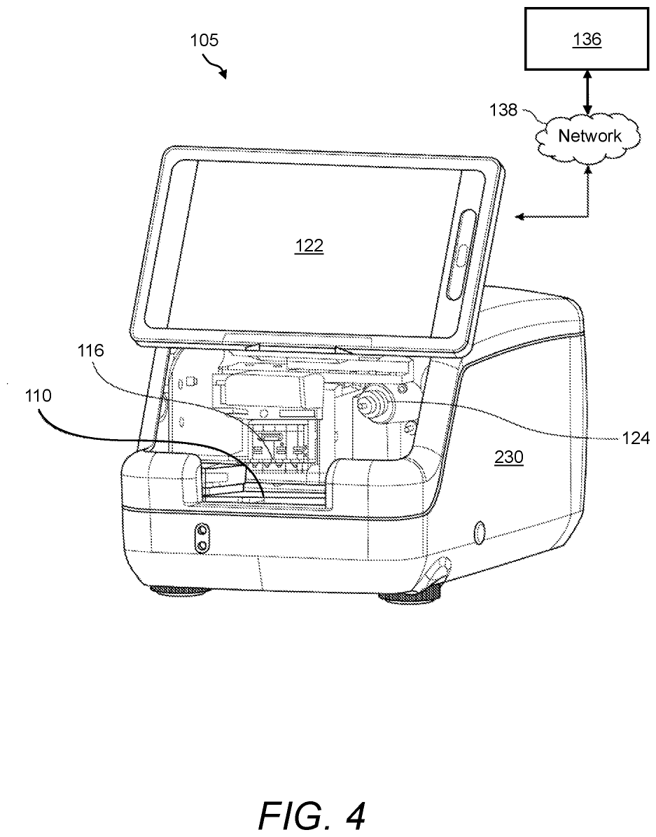

Magnets 120 may be, for example, permanent magnets and/or electromagnets. In the case of electromagnets, controller 112 may be used to control the electromagnets 120. GUI 122 may be any type of digital display for conveying information to a user. For example, GUI 122 may be the display of instrument 105 (see FIG. 4) or the display of a mobile device connected to the instrument via network 138.

Together, cartridge 110, controller 112, interface 114, detection system 116, thermal control electronics 118, magnets 120, and GUI 122 may comprise instrument 105. Optionally, instrument 105 may be connected to a network. For example, a communications interface (not shown) of controller 112 may be in communication with a networked computer 136 via a network 138. Networked computer 136 may be, for example, any centralized server or cloud-based server. Network 138 may be, for example, a local area network (LAN) or a wide area network (WAN) for connecting to the internet. The communications interface (not shown) of controller 112 may be any wired and/or wireless communication interface for connecting to a network (e.g., network 138) and by which information may be exchanged with other devices connected to the network. Though FIG. 1 shows a single networked computer 136, multiple computers (physical or virtual) may be connected in the culture monitoring system 100 via network 138. For example, one computer may be used as the thermal control mechanism 130, another computer may be used to control agitation mechanism 132, and another computer that is optimized for storing information may be used to store data received from the detection system 116, and yet another computer that is optimized for processing data may be used to process information received from instrument 105.

Controller 112 may, for example, be a general-purpose computer, special purpose computer, personal computer, tablet device, smartphone, smartwatch, mobile device, microprocessor, or other programmable data processing apparatus. Controller 112 may provide processing capabilities, such as storing, interpreting, and/or executing software instructions, as well as controlling the overall operations of culture monitoring system 100. The software instructions may comprise machine-readable code stored in non-transitory memory that is accessible by the controller 112 for the execution of the instructions. Controller 112 may be configured and programmed to control data and/or power aspects of culture monitoring system 100. Further, data storage (not shown) may be built into or provided separately from controller 112.

Further, in some embodiments, controller 112 may be external to instrument 105 (not shown in FIG. 1). The functions described above may be done remotely, for example via a mobile application running on a mobile device connected to various components (i.e., thermal controls electronics 118, among others) via a local network or another network. Output from detection 116 may also be sent to an external controller 112 via such networks and displayed on the mobile application or another mobile app running on a mobile device-specific for the external controller 112.

Generally, controller 112 may be used to manage any functions of culture monitoring system 100. For example, controller 112 may be used to manage the operations of detection system 116, thermal control electronics 118, magnets 120, GUI 122, and any other instrumentation (not shown) in relation to cartridge 110. Further, with respect to cartridge 110, controller 112 may control droplet manipulation by activating/deactivating electrodes. Further, controller 112 may be used, for example, to authenticate cartridge 110, to verify that cartridge 110 is not expired, to confirm any health conditions of cartridge 110 by running a certain protocol for that purpose, and so on.

In other embodiments of culture monitoring system 100, the functions of controller 112, detection system 116, thermal control electronics 118, magnets 120, GUI 122, and/or any other instrumentation may be integrated directly into cartridge 110 rather than provided separately from cartridge 110.

In various embodiments, the presently disclosed culture monitoring system 100 provides a cartridge 110 that may support automated processes to manipulate, process, and/or analyze biological materials. For example, cartridge 110 may support an automated process with respect to substantially continuously monitoring a standard culture bottle in a sepsis diagnostic application.

For example, instrument 105 may be designed to receive and hold a standard culture bottle 124 (e.g., a standard blood culture bottle). It should be noted that the bottle need not be necessarily embedded within the instrument but can be positioned within an accessory attached to the instrument or even be positioned within a standard culture bottle incubator. Additionally, the culture bottle can be monitored for pathogen growth through the routinely used fluorescence measurements integrated within the instrument or within the accessory or just using the standard incubator's fluorescence. Culture bottle 124 may hold a sample 126 that may include a subject's blood sample (or blood fraction or other physiologically derived samples such as swabs, urine, cerebrospinal fluid) along with certain growth media for culturing microorganisms (e.g., bacterial cells) that may be present in the blood sample. Culture bottle 124 is one example of an “incubation chamber” that may be used for culturing microorganisms (e.g., bacterial cells) that may be present in a sample.

Accordingly, culture monitoring system 100 may further include a thermal control mechanism 130, an agitation mechanism 132, and a pumping mechanism 134 arranged with respect to culture bottle 124. Thermal control mechanism 130 may be thermally coupled to culture bottle 124 for holding sample 126 at a desired temperature. Agitation mechanism 132 may be mechanically coupled to culture bottle 124 for maintaining a homogeneous sample 126 via its agitating, shaking, and/or mixing action. A liquid interface 128 may be provided for fluidly coupling culture bottle 124 to cartridge 110. In one example, liquid interface 128 may be a flexible plastic tube. Various techniques may be used for compelling flow between culture bottle 124 and cartridge 110. In one example, pressure may be used via pumping mechanism 134, which may be, for example, a peristaltic pump. In another example, the gravity-fed flow may occur between culture bottle 124 and cartridge 110. That is, the culture bottle 124 is directly interfaced with the cartridge 110 without the need for a pump to pump the culture bottle contents into the cartridge 110. Gravity allows the contents to flow directly into the cartridge 110 while a valve (not shown) controls the timing of such flow/dispensing into the cartridge 110. In this example, the standard culture bottle 124 may be modified to include a vent (not shown). Other modifications to the standard culture bottle 124 may be required depending on the type of flow between culture bottle 124 and cartridge 110.

Using culture monitoring system 100, instrument 105, and/or cartridge 110 in a sepsis diagnostic application, certain functions are required at cartridge 110. For example, cartridge 110 may support via DMF technology a sample prep 140, a stage 1 PCR 150, and a stage 2 PCR 160. In this context, “PCR” means any DNA/RNA detection method including isothermal methods, e.g., thermal cycling.

Sample prep 140 may include, but is not limited to, sample purification processes, sample lysis and binding processes, sample concentration processes, and/or sample amplification processes that may be performed via DMF technology. More details of sample prep 140 are shown and described hereinbelow with reference to FIG. 2 through FIG. 7B. Sample lysis may include chemical or physical methods.

Stage 1 PCR 150 may include but is not limited to, broad-range 16S PCR processes that may be performed via DMF technology for the detection of bacterial pathogens. More details of stage 1 PCR 150 are shown and described hereinbelow with reference to FIG. 2 through FIG. 7B.

Stage 2 PCR 160 may include, for example, certain molecular PCR processes, such as, but not limited to, molecular ID PCR processes, molecular AST PCR processes, phenotypic AST PCR processes, and PCR processes for determining whether the bacteria are Gram-positive or Gram-negative. More details of stage 2 PCR 160 are shown and described hereinbelow with reference to FIG. 2 through FIG. 7B.

Further, controller 112 may include a PCR results algorithm 190 for processing the information from detection system 116 with respect to sample prep 140, stage 1 PCR 150, and stage 2 PCR 160. For example, PCR results algorithm 190 may be used to analyze the PCR curves to determine whether sample 126 is positive for bacteria (presence) or negative for bacteria (absence) and then report it out to the user.

The operation of culture monitoring system 100, instrument 105, and/or cartridge 110 for screening for bacterial sepsis may be described generally as follows. Sample 126 which includes a sample (e.g., a blood sample) and culture medium is provided in culture bottle 124. A certain volume of sample 126 may be dispensed from culture bottle 124 to sample prep 140 of cartridge 110. At sample prep 140 a sample preparation process (e.g., sample purification, sample lysis, sample concentration, and/or sample amplification) may be performed. Next, sample prep 140 delivers the processed sample 126 to stage 1 PCR 150. Next, stage 1 PCR 150 may be used to perform a broad-range 16S PCR process to determine whether any type of bacteria is present. If no bacteria are detected at stage 1 PCR 150, then the sample may be delivered to waste. If bacteria are detected at stage 1 PCR 150, then the sample may be delivered to stage 2 PCR 160. Next, stage 2 PCR 160 may be used to determine the precise type of bacteria present. Gram status PCR can also be directly used in place of 16S PCR. In one example, stage 2 PCR 160 may be used to perform a multiplex molecular ID PCR process, a molecular AST PCR process and/or phenotypic AST PCR process, and PCR processes for determining whether the bacteria are gram-positive or gram-negative. Next, PCR results algorithm 190 may be used to analyze the PCR curves of stage 1 PCR 150 and/or stage 2 PCR 160 to determine whether sample 126 is positive or negative for bacteria and then report it to the user.

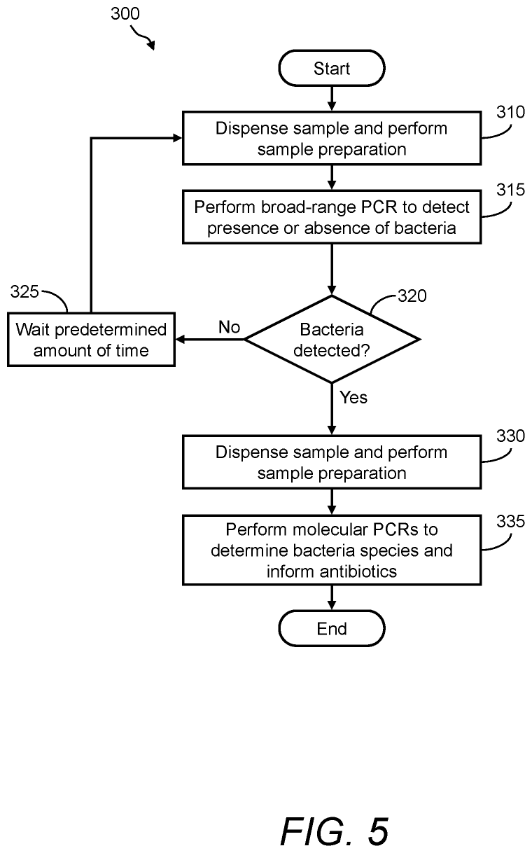

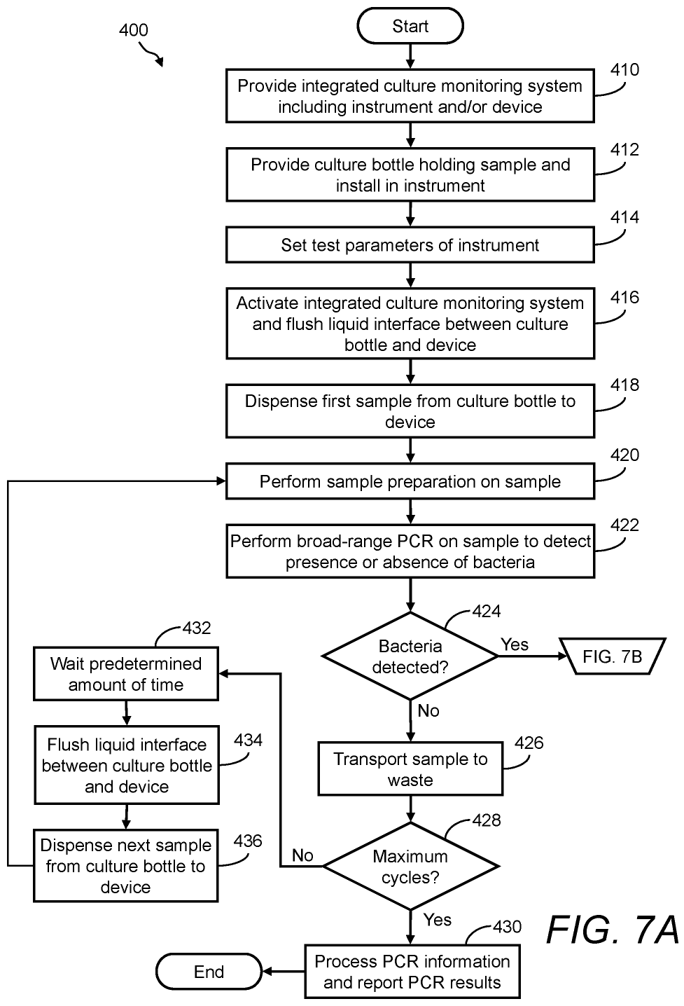

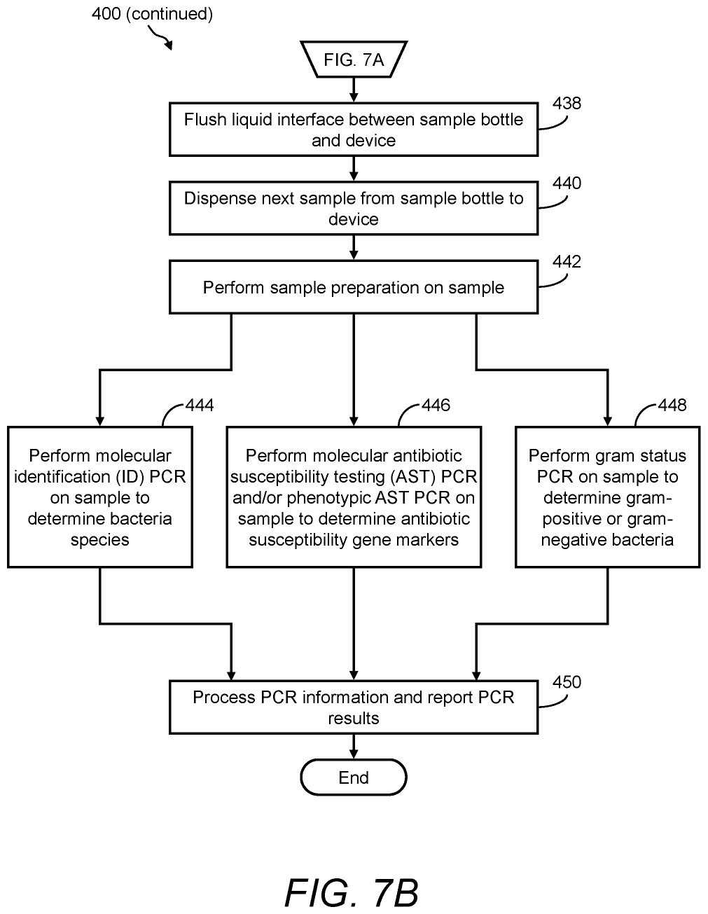

In a substantially continuous blood culture monitoring process of DMF culture monitoring system 100, a sample 126 may be dispensed from culture bottle 124 to cartridge 110, for example, from about every two (2) minutes to about every thirty (30) minutes, and then run sample prep 140, and then run stage 1 PCR 150 and/or stage 2 PCR 160 to determine whether sample 126 is positive or negative for bacteria. More details of an example of a method of using culture monitoring system 100 with respect to substantially continuously monitoring a standard blood culture bottle in a sepsis diagnostic application are shown and described herein below with reference to FIG. 5 and FIG. 7A and FIG. 7B.

Referring now to FIG. 2 is a block diagram of an example of cartridge 110 of culture monitoring system 100 shown in FIG. 1. Generally, a device may consist of two substrates (not shown) separated by a gap that forms a chamber in which the droplet operations are performed. “Droplet operation” means any manipulation of a droplet on a device, cartridge, droplet actuator, and the like. A droplet operation may, for example, include: loading a droplet into the droplet actuator; dispensing one or more droplets from a source droplet; splitting, separating, or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing.

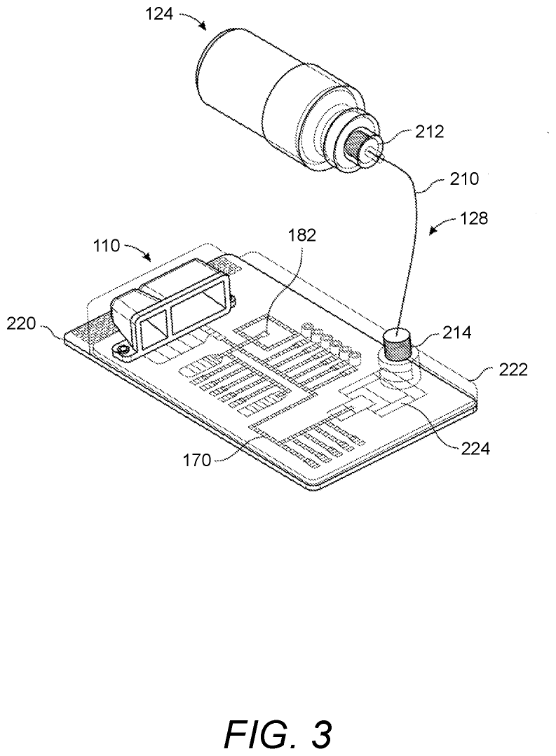

In one example, a device may include a printed circuit board (PCB) substrate (see FIG. 3) and a glass or plastic substrate separated (see FIG. 3) by a gap. Again, cartridge 110 may include sample prep 140, stage 1 PCR 150, and stage 2 PCR 160. In one example, sample prep 140 may be the portion of cartridge 110 that may be used to perform one or more sample purification processes 142, one or more sample lysis processes 144, and/or one or more sample concentration processes 146. Sample prep 140 may be used for processing sample 126 from culture bottle 124.

In one example, stage 1 PCR 150 may be the portion of cartridge 110 that may be used to perform broad-range 16S PCR 152. Amplification of the 16S rRNA gene in a PCR assay results in the detection of substantially all bacteria in a sample (i.e., amplification of the 16S rRNA gene is a pan-bacterial assay). Broad-range 16S PCR 152 may be used to determine whether any type of bacteria is present in sample 126.

In one example, stage 2 PCR 160 may be the portion of cartridge 110 that may be used to determine the precise type and/or species of bacteria present in sample 126 using molecular PCRs either as single-plex or multiplex or multiple multiplex reactions. For example, stage 2 PCR 160 may include a molecular ID PCR 162, a molecular AST PCR/phenotypic AST PCR 164, and a gram status PCR 166, which may be used to determine the type and/or species of bacteria present in the sample 126. It is important to note that phenotypic AST PCR can be used in place of molecular AST PCR or in addition to molecular AST PCR.

In one example, molecular ID PCR 162 may be designed to detect a panel of five (5) species of bacteria. For example, molecular ID PCR 162 may be designed to detect Escherichia coli (E. coli), Streptococcus agalactiae (S. agalactiae (GBS)), Staphylococcus aureus (S. aureus), Staphylococcus epidermidis (S. epidermidis), and Klebsiella pneumoniae (K. pneumoniae). However, molecular ID PCR 162 is not limited to detecting these types and the number of bacteria species only. Molecular ID PCR 162 may be designed to detect any number and species of bacteria, viruses, fungi, parasites, and any other pathogens. An ID panel for molecular ID PCR 162 may be selected based on the prevalence of certain pathogens in a population. In one example, an ID panel may be designed to detect pathogens that may be prevalent in neonatal and pediatric populations.

In one example, molecular AST PCR/phenotypic AST PCR 164 may be designed to detect three (3) antibiotic susceptibility gene markers. Knowing a certain gene may be used to inform the type of antibiotic to be used. For example, molecular AST PCR/phenotypic AST PCR 164 may be designed to detect mec A/C (e.g., for resistance to methicillin, penicillin, and other penicillin-like antibiotics), aminoglycoside resistance genes (e.g., for resistance to streptomycin, kanamycin, tobramycin, gentamicin, and neomycin), and genes associated with resistance to carbapenem antibiotics (e.g., genes associated with carbapenem-resistant Enterobacteriaceae (CRE)). However, molecular AST PCR/phenotypic AST PCR 164 is not limited to detecting these types and the number of susceptibility gene markers only. Molecular AST PCR/phenotypic AST PCR 164 may be designed to detect any type and number of susceptibility gene markers. An AST panel of susceptibility gene markers may be selected based on common resistance genes associated with the microorganisms (e.g., bacteria) detected by molecular ID PCR 162.

In one example, Gram status PCR 166 may be designed to determine whether the bacteria are Gram-positive or Gram-negative.

Referring now again to FIG. 2, sample prep 140, stage 1 PCR 150, and stage 2 PCR 160 of cartridge 110 may be DMF-based functions. Accordingly, cartridge 110 may include various other components for forming and/or supporting sample prep 140, stage 1 PCR 150, stage 2 PCR 160, and/or any other functions and/or processes of cartridge 110. For example, cartridge 110 may further include any lines, paths, and/or arrays of droplet operations electrodes 170 for forming any number and configurations of electrode arrangements 172, any number and configurations of reaction chambers 174, any number and configurations of fluid sources 176, any number and configurations of sensing mechanisms 178, any number and configurations of thermal control mechanisms 180, any number and configurations of detection spots 182, and the like.

Accordingly, droplet operations electrodes 170 may be electrowetting electrodes that are used to form any electrode arrangements 172 of cartridge 110. For example, the one or more reaction chambers 174 may be supplied by any arrangements (e.g., lines, paths, arrays) of droplet operations electrodes 170. Further, any droplet operations gap of cartridge 110 (e.g., the reaction chambers 174) may be filled with a filler fluid (not shown). The filler fluid may be a non-conductive immiscible fluid, such as a gas (e.g., air) or a liquid (e.g., an oil). Example oils may include silicone oil, hexane, and perfluorinated liquids.

Further, the one or more reaction chambers 174 and arrangements of droplet operations electrodes 170 of cartridge 110 may be supplied by any arrangements of fluid sources 176. Fluid sources 176 may be any fluid sources integrated with or otherwise fluidly coupled to cartridge 110. Fluid sources 176 may include any number and/or arrangements of, for example, on-cartridge reservoirs, off-cartridge reservoirs, blister packs, fluid ports, and the like, and any combinations thereof. In one example, cartridge 110 may include on-cartridge reservoirs only, a separate fluid source (e.g., separate reagents module) only, or both on-cartridge reservoirs and a separate fluid source.

Fluid sources 176 support any operations of cartridge 110. For example, fluid sources 176 may supply reagents needed for any sample purification processes 142, sample lysis and binding processes 144, and/or sample concentration processes 146 of sample prep 140. Further, fluid sources 176 may supply reagents needed for broad-range 16S PCR 152 of stage 1 PCR 150 or for reverse transcription in case of RNA pathogens. Further, fluid sources 176 may supply reagents and/or specific probes/primers needed for molecular ID PCR 162, molecular AST PCR/phenotypic AST PCR 164, and/or Gram status PCR 166 of stage 2 PCR 160. Additionally, certain reagents may be dried reagents that are reconstituted on cartridge 110 at runtime. Accordingly, fluid sources 176 may supply the liquid needed to support any reconstitution processes.

Sensing mechanisms 178 of cartridge 110 may be any components and/or elements built into cartridge 110 to support any feedback mechanisms, such as impedance or capacitance sensing. For example, sensors may be embedded at each droplet operations electrode 170 location to measure impedance, which enables monitoring and closed-loop control of certain droplet operations. Examples of other types of sensors may include temperature sensors, optical sensors, electrochemical sensors, voltage sensors, and current sensors. Sensing mechanisms 178 may be driven and/or controlled by controller 112, or a mobile device connected to instrument 105 via network 138.

Thermal control mechanisms 180 of cartridge 110 may be any components and/or elements built into cartridge 110 for holding cartridge 110 or any portion thereof at a certain temperature. For example, thermal control mechanisms 180 may be resistive heaters and/or thermoelectric (e.g., Peltier) devices integrated directly within cartridge 110 and/or arranged externally in thermal contact with cartridge 110. Thermal control mechanisms 180 may be driven and/or controlled using thermal control electronics 118 of controller 112, or a mobile device connected to instrument 105 via network 138.

Detection spots 182 of cartridge 110 may be any droplet operations electrodes 170 designated for detection operations via detection system 116. For example, in optical detection, an illumination source and an optical measurement device of detection system 116 may be provided in relation to a certain detection spot 182 at which a droplet to be analyzed may be parked. Certain detection spots 182 may be associated with sample prep 140. Certain other detection spots 182 may be associated with stage 1 PCR 150. Certain other detection spots 182 may be associated with stage 2 PCR 160. Multiple distinct species may be measured at each detection spot. The entire cartridge may be a detection spot when a camera is used to image the entire cartridge.

Referring now to FIG. 3 is a perspective view of an example of cartridge 110 and culture bottle 124 of culture monitoring system 100 shown in FIG. 1. Culture bottle 124 may be fluidly coupled to cartridge 110 via liquid interface 128. In one example, liquid interface 128 may include a flow line 210 arranged between a bottle coupling 212 and a DMF coupling 214. Flow line 210 may be, for example, a length of flexible plastic tubing. Bottle coupling 212 may be designed to mate with culture bottle 124. Bottle coupling 212 may be, for example, a rubber or plastic bottle stopper or cap into which one end of the flow line 210 is integrated. Coupling 214 may be designed to mate with a fluid port of cartridge 110. Coupling 214 may be, for example, a rubber or plastic coupler into which the other end of the flow line 210 is integrated.

Additionally, FIG. 3 shows an example of cartridge 110. For example, cartridge 110 may be formed of a bottom substrate 220 and a top substrate 222 separated by a gap. Bottom substrate 220 may be, for example, a PCB. Top substrate 222 may be, for example, a substantially transparent glass or plastic substrate. An arrangement of droplet operations electrodes 170 and reservoir electrodes 224 may be provided on bottom substrate 220. Also shown is an example of a detection spot 182 on bottom substrate 220. Reservoir electrodes 224 are provided to support an on-cartridge reservoir.

Referring now to FIG. 4 is a perspective view of an example of instrument 105 of culture monitoring system 100 shown in FIG. 1. Instrument 105 may include an instrument housing 230 for holding all components thereof. While all its components are not visible, FIG. 4 shows an example of cartridge 110, detection system 116, GUI 122, and culture bottle 124 installed with respect to instrument housing 230 of instrument 105. Culture bottle 124 can also be installed in an “accessory” that is connected to instrument 105. Multiple such culture bottle accessories (including incubation and mechanical agitation) can be connected to a single instrument 105, which itself can handle multiple cartridges, or to multiple instruments 105. Further, multiple cartridges can be loaded into a single instrument 105, or into multiple bays of a single instrument 105, or into multiple instruments 105, that are modularly connected to form a larger instrument. Irrespective of detecting a positive or a negative pathogenic presence in the cartridge for a specimen that is sampled from the culture bottle, the bottle may stay within the accessory for a longer time. More importantly, when the sampling does not show any positive pathogenic presence in the first few hours, the bottle will go on to complete incubation for up to several days in the accessory to ensure that any slow-growing pathogens are identified. These bottles will be available for further testing after completion of such a long duration of incubation. During this long incubation time, the bottle is disconnected from the cartridge while another bottle is loaded into the accessory and connected to a cartridge to initiate rapid culture sampling. Traditional fluorescence detection may also be available in instrument 105 and the accessory so pathogens that are not included in the cartridge detection test panel can be detected during long incubation times.

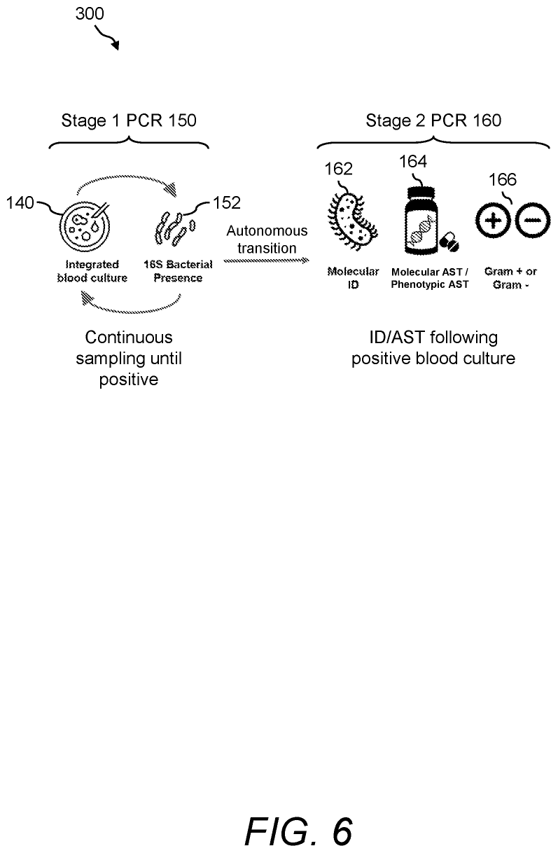

Referring now to FIG. 5 is a flow diagram of an example of a method 300 of using the culture monitoring system 100 shown in FIG. 1 according to a simple configuration, which is an example of a method of using the presently disclosed integrated culture monitoring system including an instrument and/or device. Further, FIG. 6 depicts pictorially the method 300 shown in FIG. 5. Method 300 may include, but is not limited to, the following steps.

At a step 310, a (first) sample (or a first portion of the first sample) is dispensed, and a sample preparation process is performed on the sample. For example, sample 126 may be dispensed from culture bottle 124 to the sample prep 140-portion of cartridge 110. In one example, sample 126 is a blood sample for screening for bacterial sepsis. Then, a sample preparation process may be performed using, for example, certain purification processes 142, sample lysis processes 144, and/or sample concentration processes 146 of sample prep 140. Method 300 may proceed to step 315.

At a step 315, a broad-range PCR is performed on the sample to detect the presence or absence of any type of bacteria. For example, the processed sample 126 may be passed from sample prep 140 to stage 1 PCR 150. At stage 1 PCR 150, a broad-range 16S PCR may be performed to determine whether any type of bacteria is present. Method 300 may proceed to step 320.

At a decision step 320, it is determined whether bacteria are detected in sample 126 based on the results of stage 1 PCR 150 at step 315. If bacteria are not detected, then method 300 may proceed to step 325. However, if bacteria are detected, then method 300 may proceed to step 330.

At a step 325, the method waits a predetermined amount of time. For example, method 300 may wait from about two (2) minutes to about thirty (30) minutes and then return to step 310 for the next sample dispense and preparation operation.

At a step 330, a (second) sample (or a second portion of the first sample) is dispensed, and a sample preparation process is performed on the sample. For example, sample 126 may be dispensed from culture bottle 124 to the sample prep 140-portion of cartridge 110. Then, a sample preparation process may be performed using, for example, certain purification processes 142, sample lysis processes 144, and/or sample concentration processes 146 of sample prep 140. Optionally, instead of performing another sample dispense operation here, if there is enough material remaining from the first sample prep at step 310 and the 16S PCR test at step 315 is positive, then a remaining aliquot of the prepared sample at step 310 may be provided to step 335. Method 300 may proceed to step 335.

At a step 335, certain molecular PCRs are performed to determine the bacteria species and to inform the antibiotics to combat the infection. For example, molecular ID PCR 162 of stage 2 PCR 160 may be used to test for certain bacteria species, such as, but not limited to, E. coli, S. agalactiae (GBS), S. aureus, S. epidermidis, and Klebsiella pneumoniae. Further, molecular AST PCR 164 of stage 2 PCR 160 may be used to test for certain antibiotic susceptibility gene markers (e.g., antibiotic resistance markers), such as but not limited to, mec A/C (e.g., for methicillin, penicillin, and other penicillin-like antibiotics), aminoglycoside (e.g., streptomycin, kanamycin, tobramycin, gentamicin, and neomycin), and carbapenem-resistant Enterobacteriaceae (CRE). Further, gram status PCR 166 of stage 2 PCR 160 may be used to determine whether the detected bacteria are gram-positive or gram-negative. Method 300 ends.

Referring now to FIG. 7A and FIG. 7B is a flow diagram of an example of a method 400 of using the culture monitoring system 100 shown in FIG. 1, which is an example of a method of using the presently disclosed integrated culture monitoring system including an instrument and/or device. For example, method 400 may be used to substantially continuously monitor a standard blood culture bottle using DMF-based technology. Method 400 may include but is not limited to, the following steps.

At a step 410, an integrated culture monitoring system including an instrument and/or device is provided. For example, culture monitoring system 100 including instrument 105 and cartridge 110 as described hereinabove with reference to FIG. 1 through FIG. 4 may be provided. Additionally, all liquids needed to support the operations of instrument 105 and/or cartridge 110 are provided. For example, reagents needed to support any sample purification processes 142, sample lysis processes 144, and/or sample concentration processes 146 of sample prep 140 may be provided. Further, reagents needed to support broad-range 16S PCR 152 of stage 1 PCR 150 may be provided. Further, reagents and/or specific probes/primers needed to support molecular ID PCR 162, molecular AST PCR/phenotypic AST PCR 164, and/or gram status PCR 166 of stage 2 PCR 160 may be provided. Additionally, certain reagents may be dried reagents that are reconstituted at cartridge 110 at runtime or prior to runtime. Method 400 may proceed to step 412.

At a step 412, the culture bottle holding the sample is provided and installed in the instrument. For example, culture bottle 124 holding sample 126 may be provided and installed in instrument 105 or outside the instrument 105 in an accessory. Method 400 may proceed to step 414.

At a step 414, the test parameters of instrument 105 are set. For example, the dispense/test interval and/or total process time may be set. In one example, the dispense/test interval may be set from about two (2) minutes to about thirty (30) minutes and the total process time may be set to about eight (8) hours, about six (6) hours, about four (4) hours, about two (2) hours, or about one (1) hour. In this example, the total number of dispense/test cycles of instrument 105 is thirteen (13). In another example, the dispense/test interval may be set from about two (2) minutes to about thirty (30) minutes and the total process time may be set to twenty-four (24) hours. In this example, the total number of dispense/test cycles of instrument 105 is forty-nine (49). Method 400 may proceed to step 416.

At a step 416, the integrated culture monitoring system is activated and the liquid interface between the culture bottle and the device is flushed. For example, culture monitoring system 100 may be activated and liquid interface 128 between culture bottle 124 and cartridge 110 may be flushed. In one example, if flow line 210 of liquid interface 128 holds about 40 μL of liquid, then about 40 μL of liquid may be flushed out of flow line 210 and into a waste reservoir of cartridge 110 so that a fresh quantity of sample 126 may be dispensed from culture bottle 124. Method 400 may proceed to step 418.

At a step 418, a (first) sample (or a first portion of the first sample) is dispensed from the culture bottle to the device. For example, a first quantity of sample 126 may be dispensed from culture bottle 124 to the sample prep 140-portion of cartridge 110. In one example, about 1 μL to about 10 μL of sample 126 may be dispensed. In another example, for pathogens available at low copy numbers or CFU or concentration, as much as 1 mL may be dispensed with further sample concentration performed within the cartridge. Method 400 may proceed to step 420.

At a step 420, a sample preparation process is performed on the sample. For example, a sample preparation process may be performed using, for example, certain purification processes 142, sample lysis processes 144, and/or sample concentration processes of sample prep 140. Method 400 may proceed to step 422.

At a step 422, a broad-range PCR is performed on the sample to detect the presence or absence of any type of bacteria. For example, the processed sample 126 may be passed from sample prep 140 to stage 1 PCR 150. At stage 1 PCR 150, broad-range 16S PCR 152 may be performed to determine whether any type of bacteria is present. Method 400 may proceed to step 424.

At a decision step 424, it is determined whether bacteria are detected in sample 126 based on the results of stage 1 PCR 150 at step 422. If bacteria are not detected, then method 400 may proceed to step 426. However, if bacteria are detected, then method 400 may proceed to step 438.

At step 426, the sample may be transported to waste. For example, sample 126 may be transported to a waste reservoir of cartridge 110. Method 400 may proceed to step 428.

At a decision step 428, it is determined whether the maximum number of dispense/test cycles has been reached. In one example, if the number of maximum dispense/test cycles of instrument 105 is set in step 414 to thirteen (13), then it is determined whether thirteen (13) dispense/test cycles have been completed. In another example, if the number of maximum dispense/test cycles of instrument 105 is set in step 414 to forty-nine (49), then it is determined whether forty-nine (49) dispense/test cycles have been completed. If the number of maximum dispense/test cycles has been reached, then method 400 may proceed to step 430. However, if the number of maximum dispense/test cycles has not been reached, then method 400 may proceed to step 432.

At a step 430, the PCR information is processed, and the PCR results are reported. For example, PCR results algorithm 190 may be used to analyze the PCR curves of broad-range 16S PCR 152 of stage 1 PCR 150 and then report the PCR results out to the user. For example, the PCR results may be displayed at GUI 122 of instrument 105. In one example, the PCR results may be displayed in real-time throughout the steps of method 400. In another example, the PCR results may be displayed at the end of method 400. Method 400 ends.

At a step 432, the method waits a predetermined amount of time. For example, method 400 may wait for the dispense/test interval set in step 414. In one example, method 400 may wait from about two (2) minutes to about thirty (30) minutes and then proceed to step 434.

At a step 434, the liquid interface between the culture bottle and the device is flushed. For example, liquid interface 128 between culture bottle 124 and cartridge 110 may be flushed of any liquid held therein. In one example, if flow line 210 of liquid interface 128 holds about 40 μL of liquid, then about 40 μL of liquid may be flushed out of flow line 210 and into a waste reservoir of cartridge 110 so that a fresh quantity of sample 126 may be dispensed from culture bottle 124. Method 400 may proceed to step 436.

At a step 436, a next (second) sample (or a first portion of the second sample or a second portion of the first sample) is dispensed from the culture bottle to the device. For example, a next quantity of sample 126 may be dispensed from culture bottle 124 to the sample prep 140-portion of cartridge 110. In one example, about 1 μL to about 10 μL of sample 126 may be dispensed. Method 400 may return to step 420.

At a step 438, the liquid interface between the culture bottle and the DMF device is flushed. For example, liquid interface 128 between culture bottle 124 and cartridge 110 may be flushed of any liquid held therein. In one example, if flow line 210 of liquid interface 128 holds about 40 μL of liquid, then about 40 μL of liquid may be flushed out of flow line 210 and into a waste reservoir of cartridge 110 so that a fresh quantity of sample 126 may be dispensed from culture bottle 124. Method 400 may proceed to step 440.

At a step 440, a next (third) sample (or a second portion of the second sample or a third portion of the first sample) is dispensed from the culture bottle to the device. For example, a next quantity of sample 126 may be dispensed from culture bottle 124 to the sample prep 140-portion of cartridge 110. In one example, about 1 μL to about 10 μL of sample 126 may be dispensed. Method 400 may return to step 442.

At a step 442, a sample preparation process is performed on the sample. For example, a sample preparation process may be performed using, for example, certain purification processes 142, sample lysis processes 144, and/or sample concentration processes 146 of sample prep 140. The method may proceed to steps 444, 446, and 448 in parallel.

At a step 444, a molecular ID PCR is performed on the sample to determine the bacteria species. For example, the processed sample 126 may be passed from sample prep 140 to stage 2 PCR 160. At stage 2 PCR 160, molecular ID PCR 162 may be performed to determine the bacteria species. For example, molecular ID PCR 162 of stage 2 PCR 160 may be used to test for certain bacteria species, such as, but not limited to, E. coli, S. agalactiae (GBS), S. aureus, S. epidermidis, and Klebsiella pneumoniae. Method 400 may proceed to step 450.

At a step 446, a molecular AST PCR is performed on the sample to determine antibiotic susceptibility gene markers. For example, the processed sample 126 may be passed from sample prep 140 to stage 2 PCR 160. At stage 2 PCR 160, molecular AST PCR 164 may be performed to determine antibiotic susceptibility gene markers. For example, molecular AST PCR 164 of stage 2 PCR 160 may be used to test for certain antibiotic susceptibility gene markers, such as, but not limited to, mec A/C (e.g., for methicillin, penicillin, and other penicillin-like antibiotics), aminoglycoside (e.g., streptomycin, kanamycin, tobramycin, gentamicin, and neomycin), and carbapenem-resistant Enterobacteriaceae (CRE). Method 400 may proceed to step 450.

At a step 448, a gram status PCR is performed on the sample to determine gram-positive or gram-negative bacteria. For example, the processed sample 126 may be passed from sample prep 140 to stage 2 PCR 160. At stage 2 PCR 160, Gram status PCR 166 may be performed to determine Gram-positive or Gram-negative bacteria. Method 400 may proceed to step 450.

At step 450, the PCR information is processed, and the PCR results are reported. For example, PCR results algorithm 190 may be used to analyze the PCR curves of broad-range 16S PCR 152 of stage 1 PCR 150, molecular ID PCR 162 of stage 2 PCR 160, molecular AST PCR/phenotypic AST PCR 164 of stage 2 PCR 160, and/or gram status PCR 166 of stage 2 PCR 160 and then report the PCR results out to the user. For example, the PCR results may be displayed at GUI 122 of DMF instrument 105. In one example, the PCR results may be displayed in real-time throughout the steps of method 400. In another example, the PCR results may be displayed at the end of method 400. Method 400 ends.

### Phenotypic AST PCR Assay

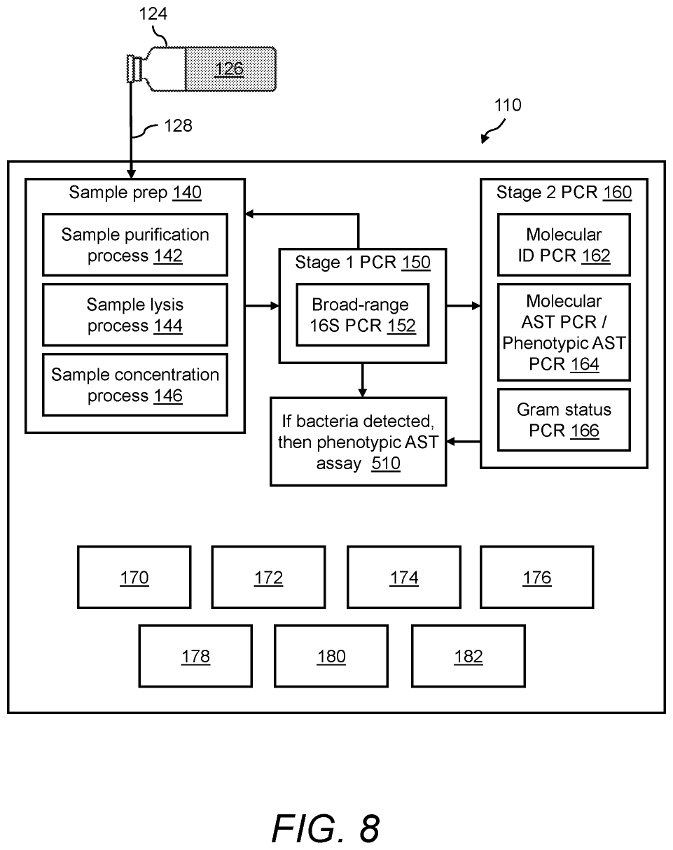

Referring now to FIG. 8 is a block diagram of another example of cartridge 110 of the culture monitoring system 100 shown in FIG. 1. In this example, cartridge 110 further supports phenotypic AST processes, such as a phenotypic AST PCR assay 510. In one example, phenotypic AST PCR assay 510 may be provided along with stage 2 PCR 160. In another example, phenotypic AST PCR assay 510 may be provided in place of stage 2 PCR 160.

As compared with stage 2 PCR 160, phenotypic AST assay 510 may be used to achieve a yet higher quality of antimicrobial susceptibility testing by growing the bacteria in cartridge 110 itself in the presence of certain antibiotics. When using phenotypic AST PCR assay 510, sample prep 140 may exclude sample lysis processes 144 and run purification processes 142 and/or concentration processes 146 only. Sample lysis processes 144 may be excluded, e.g., because the cells need to be kept alive.

In one example, phenotypic AST assay 510 may include three- (3) to twenty (20) different antibiotics on cartridge 110 at up to six (6) different concentrations for each antibiotic plus a zero-concentration control. Then, the sample (most of the time it is already in the culture medium) may be split into twenty-one (21) to one hundred and forty (140) different droplets that may be reacted with the three (3) to twenty (20) different antibiotics, respectively. Then, the growth of the bacteria is tracked over time through optical methods such as turbidimetry, fluorometry, or brightfield or fluorescence microscopy or through electrical methods such as impedance measurements taken at the electrode. Examples of fluorescent dyes include the lipophilic dyes SynaptoGreen C4 (Sigma, S6814) and FM1-43 (Invitrogen, T3163), and the metabolic dye fluorescein diacetate. If the bacteria do not multiply in the presence of a certain antibiotic, then those bacteria are susceptible to that antibiotic. If the bacteria do multiply in the presence of a certain antibiotic, then those bacteria are not susceptible to that antibiotic. With seven (7) different concentrations of antibiotics, minimum inhibitory concentration (MIC) curves can also be constructed.

In cartridge 110, phenotypic AST PCR assay 510 requires certain reagents as well as culture medium for growing the bacteria. The process of using phenotypic AST PCR assay 510 may require regular replenishing of the culture medium to maintain the growth of the bacterial culture. In some cases, the antibiotics that will be tested for phenotypic AST will be selected based on the Gram-positive, Gram-negative, or the specific pathogen identified. It is also possible that the phenotypic AST may be set up on the same cartridge as the identification cartridge or on a different cartridge that can accept any culture medium sample as an input, not necessarily the samples that were previously processed by the identification cartridge.

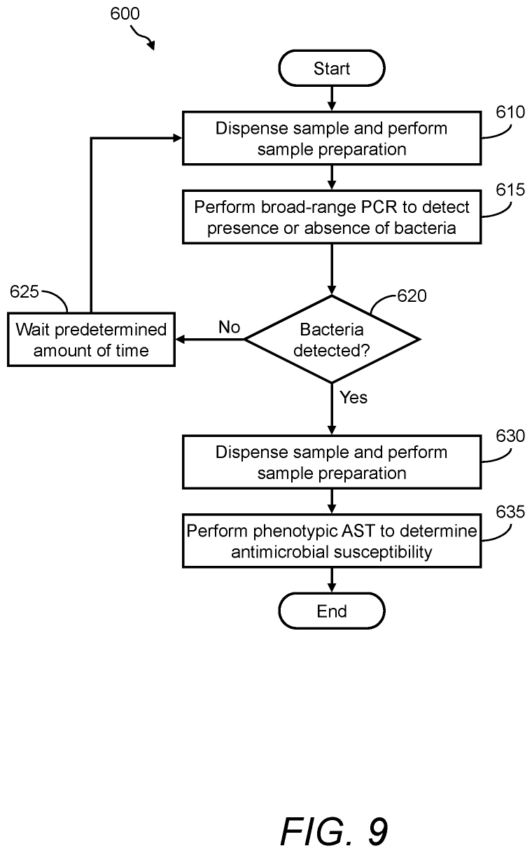

Referring now to FIG. 9 illustrates a flow diagram of an example of a method 600 of using cartridge 110 shown in FIG. 8 that includes phenotypic AST assay 510, which is another example of a method of using the presently disclosed integrated culture monitoring system including an instrument and/or device. Method 600 may include but is not limited to, the following steps.

At a step 610, a (first) sample (or a first portion of the first sample) is dispensed, and a sample preparation process is performed on the sample. For example, sample 126 may be dispensed from culture bottle 124 to the sample prep 140-portion of cartridge 110. Then, a sample preparation process may be performed using, for example, certain purification processes 142, sample lysis processes 144, and/or sample concentration processes 146 of sample prep 140. Method 600 may proceed to step 615.

At a step 615, a broad-range PCR is performed on the sample to detect the presence or absence of any type of bacteria. For example, the processed sample 126 may be passed from sample prep 140 to stage 1 PCR 150. At stage 1 PCR 150, a broad-range 16S PCR may be performed to determine whether any type of bacteria is present. Method 600 may proceed to step 620.

At a decision step 620, it is determined whether bacteria are detected in sample 126 based on the results of stage 1 PCR 150 at step 615. If bacteria are not detected, then method 600 may proceed to step 625. However, if bacteria are detected, then method 600 may proceed to step 630.

At a step 625, the method waits a predetermined amount of time. For example, method 600 may wait about two (2) minutes to about thirty (30) minutes and then return to step 610 for the next sample dispense and preparation operation.

At a step 630, a (second) sample (or a first portion of the second sample or a second portion of the first sample) is dispensed, and a sample preparation process is performed on the sample. For example, sample 126 may be dispensed from culture bottle 124 to the sample prep 140-portion of cartridge 110. Then, a sample preparation process may be performed using, for example, certain purification processes 142, and/or sample concentration processes 146 of sample prep 140. The sample preparation process may exclude sample lysis processes 144 of sample prep 140. Method 600 may proceed to step 635.

At a step 635, phenotypic AST assays are performed to determine the antimicrobial susceptibility of the bacteria. For example, phenotypic AST assay 510 may include three- (3) to twenty (20) different antibiotics on cartridge 110. Then, the sample may be split into twenty-one (21) to one hundred and forty (140) different droplets that may be reacted with the three (3) to five (5) different antibiotics, respectively at seven (7) different concentrations each. Then, the growth of the bacteria is tracked over time. In one example, an optical sensor for detecting bacteria in culture fluid may be used to track the growth of the bacteria over time. If the bacteria do not multiply in the presence of a certain antibiotic, then those bacteria are susceptible to that antibiotic. If the bacteria do multiply in the presence of a certain antibiotic, then those bacteria are not susceptible to that antibiotic. Method 600 ends.

A molecular ID test, a molecular AST test and/or phenotypic AST test, and a Gram status test may be performed concurrently on a third prepared sample, a second portion of the second prepared sample, or on a third portion of the first prepared sample.

Depending on the circumstances, the identification of bacterial species and antimicrobial susceptibility testing may both be completed within about eight (8) hours, within about (6) hours, within about four (4) hours, within about two (2) hours, or within about one (1) hour.

Further, depending on the circumstances, the detection of bacteria, the Gram status test, the ID test, and/or the AST test may be completed within about eight (8) hours, within about six (6) hours, within about four (4) hours, within about two (2) hours, or within about one (1) hour.

### Host Response Signature

A patient's (i.e., the host's) gene response signature can be used to delineate a bacterial infection from a non-bacterial (e.g., viral) infection or delineate an infection from a non-infection or further delineate infectious etiologies and their causative pathogens. In one example, a transcriptional profile in cells (e.g., immune cells) present in a blood sample can be used to determine a host response signature for diagnosing infection. Further, a patient's host response signature can be used to guide the selection of appropriate therapy (e.g., an antibiotic therapy) for treating the infection.

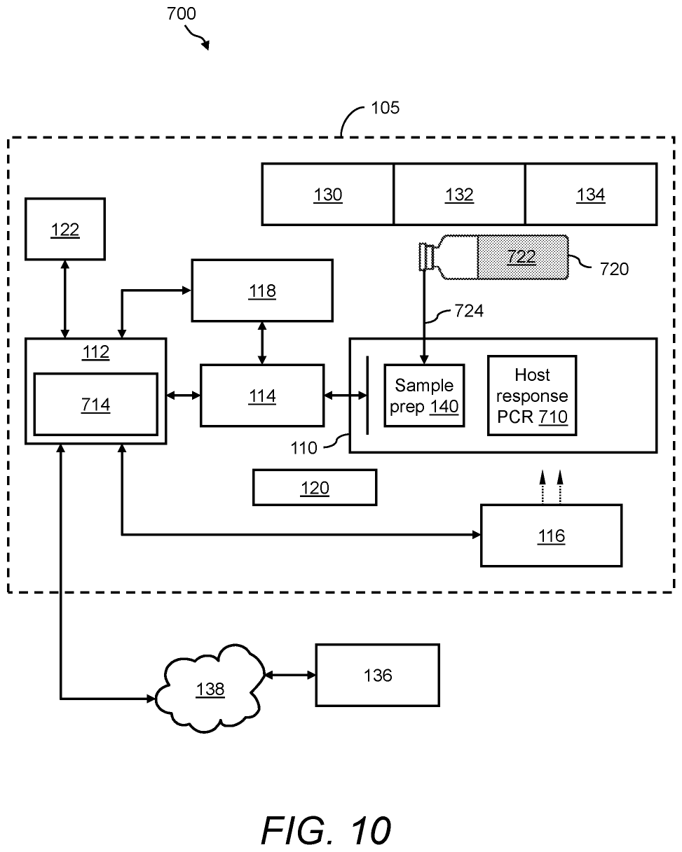

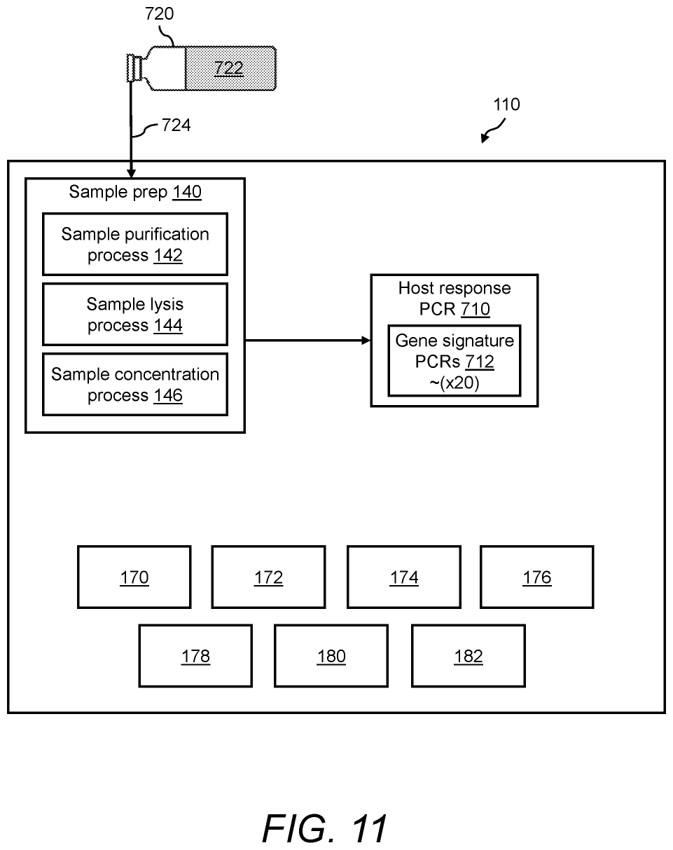

In some embodiments, the presently disclosed integrated culture monitoring system and method including an instrument and/or device may include a cartridge (or device) configured for directly processing a blood sample for determining a host response signature to delineate between a bacterial and non-bacterial (e.g., viral) infection, wherein these processes may be DMF-based processes. More details of cartridge 110 for directly processing a blood sample and determining a host response signature are described hereinbelow with reference to FIG. 10 and FIG. 11.

Referring now to FIG. 10 is a block diagram of an example of a culture monitoring system 700 that further supports host response PCR processes, which is another example of the presently disclosed integrated culture monitoring system including an instrument and/or device. Culture monitoring system 700 is substantially the same as culture monitoring system 100 shown in FIG. 1 except that sample prep 140 is configured for directly processing a sample, e.g., a blood sample. Further, stage 1 PCR 150 and stage 2 PCR 160 of cartridge 110 are replaced with a host response PCR 710. Further, PCR comprises RT-PCR and other isothermal and thermal cycling methods for amplification of DNA and CRISPR/CAS, fluorescence dyes, and other methods of DNA detection. Further, PCR results algorithm 190 of controller 112 is replaced with PCR results algorithm 714. Further, in cartridge 110 shown in FIG. 11, host response PCR 710 further includes multiple gene signature PCRs 712.

In one example, instrument 105 may be designed to receive and hold an input tube 720. Input tube 720 may hold a sample 722 (such as a blood sample, etc.) for direct processing on cartridge 110. A liquid interface 724 may be provided for fluidly coupling input tube 720 to cartridge 110.

In another example, a sample, e.g., a blood sample, may be collected in a collection tube that has stabilizers for mRNA and is then transferred to the cartridge for further processing. Alternatively, the sample (e.g., a drop of blood) may be collected or loaded directly onto the cartridge without any collection tubes.

PCR results algorithm 714 may be used for processing the information from detection system 116 with respect to sample prep 140 and host response PCR 710. For example, PCR results algorithm 714 may be used to analyze the PCR curves to determine a host response signature for diagnosing the presence of an infection, type of infection, severity of infection, and prognosis of the infection, and then report it out to the user. For example, the report may just be a probability score.

Referring now to FIG. 11 is a block diagram of an example of cartridge 110 of culture monitoring system 100 shown in FIG. 10. Again, cartridge 110 may include sample prep 140 and host response PCR 710. In one example, sample prep 140 may be the portion of cartridge 110 that may be used to perform one or more sample purification processes (isolation of peripheral blood mononuclear cells, white blood cells, and whole blood) 142, one or more sample lysis processes 144 (e.g., blood cell lysis), and/or one or more sample concentration processes 146 (e.g., the concentration of mRNA). Sample prep 140 may be used for directly processing sample 722 from input tube 720.

Gene signature PCRs 712 of host response PCR 710 may be designed for multiplexed detection of a panel of gene targets that can be used to determine a host response signature for diagnosing infection. In one example, up to thirty-five (35) gene targets may be used to delineate between a bacterial infection and a non-bacterial (e.g., viral) infection. In another example, a panel of gene targets may be selected to diagnose a specific type of bacterial infection.

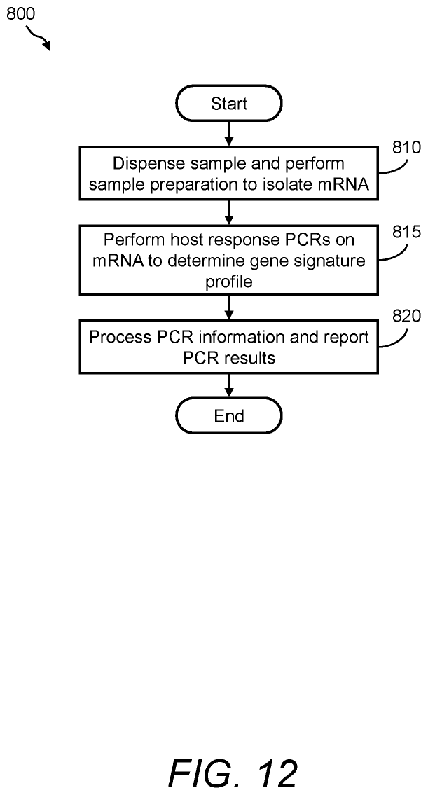

Referring now to FIG. 12 is a flow diagram of an example of a method 800 of using the culture monitoring system 700 shown in FIG. 10 that includes host response PCR processes, which is another example of a method of using the presently disclosed integrated culture monitoring system including an instrument and/or device. Method 800 may include but is not limited to, the following steps.

At a step 810, a sample is dispensed, and a sample preparation process is performed on the sample to isolate mRNA. For example, sample 722 may be dispensed from input tube 720 to the sample prep 140-portion of cartridge 110. Then, a sample preparation process may be performed using, for example, certain purification processes 142, sample lysis processes 144 (e.g., blood cell lysis), sample concentration processes 146 (e.g., the concentration of mRNA) of sample prep 140 to generate a processed (blood) sample comprising mRNA. Method 800 may proceed to step 815.

At a step 815, a host response PCR is performed on the isolated mRNA to determine a gene signature profile. For example, the processed sample 722 may be passed from sample prep 140 to host response PCR 710. At host response PCR 710, a gene signature PCRs 712 may be performed to determine the transcriptional profile of the sample. Method 800 may proceed to step 820.

At a step 820, the PCR information is processed, and the PCR results are reported. For example, PCR results algorithm 714 may be used to analyze the PCR curves of gene signature PCRs 712 of host response PCR 710 and then report the PCR results to the user. For example, the PCR results may be displayed at GUI 122 of instrument 105. In one example, the PCR results may be displayed at the end of method 800. Method 800 ends.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions, and uses of the invention will be apparent to the skilled person without departing from the scope and spirit of the invention. Although the invention has been disclosed in connection with specific preferred aspects or embodiments, the invention as claimed should not be unduly limited to such specific aspects or embodiments.

The present invention may be implemented using hardware, software, or a combination thereof and may be implemented in one or more computer systems or other processing systems. In one aspect, the invention is directed toward one or more computer systems capable of carrying out the phenotypicity described herein.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the presently disclosed subject matter.

For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Unless specifically stated otherwise, terms such as “receiving,” “routing,” “updating,” “providing,” or the like, refer to actions and processes performed or implemented by computing devices that manipulate and transform data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general-purpose computing device selectively programmed by a computer program stored in the computing device. Such a computer program may be stored in a computer-readable non-transitory storage medium.

The methods and illustrative examples described herein are not inherently related to any particular computer or other apparatus. Various general-purpose systems may be used in accordance with the teachings described herein, or it may prove convenient to construct a more specialized apparatus to perform the required method steps. The required structure for a variety of these systems will appear as set forth in the description above.

It should also be noted that in some alternative implementations, the functions/acts noted may occur out of the order noted in the figures. For example, two (2) figures shown in succession may in fact be executed substantially concurrently or may sometimes be executed in the reverse order, depending upon the phenotypicity/acts involved.

Although the method operations were described in a specific order, other operations may be performed in between described operations, described operations may be adjusted so that they occur at slightly different times, or the described operations may be distributed in a system that allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on).

The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component.

Additionally, “configured to” or “configurable to” can include generic structure (e.g., generic circuitry) that is manipulated by software and/or firmware (e.g., an FPGA or a general-purpose processor executing software) to operate in a manner that can perform the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or another unprogrammed device, unless accompanied by programmed media that confers the ability to the unprogrammed device to be configured to perform the disclosed function(s).

Although the foregoing subject matter has been described in some detail by way of illustration and example for purposes of clarity of understanding, it will be understood by those skilled in the art that certain changes and modifications can be practiced within the scope of the appended claims.

We claim:

1. An integrated culture monitoring system comprising:
(a) an incubation chamber;
(b) a temperature control device arranged to control temperature of the incubation chamber;
(c) a fluid-handling cartridge;
(d) a fluid passage operationally coupled to a pump and providing a fluid passage between:
(i) the incubation chamber; and
(ii) the fluid-handling cartridge; and
arranged to permit the flow of fluid between the incubation chamber and the fluid-handling cartridge.

2. The integrated culture monitoring system of claim 1, further comprising one or more processors:
(a) electronically coupled to the temperature control device, the fluid-handing cartridge, and the pump; and
(b) programmed to control one or more of:
(i) the temperature control device to increase or decrease temperature;
(ii) the pump to control movement of fluid through the fluid passage; and/or
(iii) dispensing and assaying of a droplet of culture fluid pumped via the fluid passage into the fluid-handling cartridge.

3. The integrated culture monitoring system of any one of claims 1 to 2, wherein the fluid-handling cartridge is a digital microfluidics (DMF) cartridge.

4. The integrated culture monitoring system of any one of the preceding claims, wherein the incubation chamber includes culture fluid.

5. The integrated culture monitoring system of claim 4, wherein the culture fluid is selected from a group consisting of whole blood, blood fractions, urine, other bodily fluids, culture media, or combinations of the foregoing.

6. The integrated culture monitoring system of any one of the preceding claims, wherein the fluid-handling cartridge comprises reagents for performing one or more assays on a sample of the culture fluid.

7. The integrated culture monitoring system of any one of the preceding claims, wherein the system further comprises one or more sensors electronically connected to and controlled by one or more system processors.

8. The integrated monitoring system of claim 7, wherein the one or more sensors are arranged to detect a reaction in a droplet on the fluid-handling cartridge.

9. The integrated culture monitoring system of any one of the preceding claim, wherein the incubation chamber is fluidly connected to the fluid-handling cartridge.

10. The integrated culture monitoring system of any one of the preceding claims, wherein the integrated culture monitoring system further comprises an agitation mechanism mechanically coupled to or otherwise contacting the incubation chamber.

11. The integrated culture monitoring system of claim 7, wherein the one or more sensors comprise an optical sensor arranged to detect bacteria in culture fluid.

12. The integrated culture monitoring system of claim 1, wherein the optical sensor is selected from a group consisting of a fluorimeter, a charge-coupled device, a photodetector, a spectrometer, a photodiode array, or any combination thereof.

13. The integrated culture monitoring system of any one of the preceding claims, wherein the fluid-handling cartridge comprises two substrates separated by a gap that forms a chamber in which droplet operations are performed.

14. The integrated culture monitoring system of any one of the preceding claims, wherein the incubation chamber comprises a culture bottle.

15. The integrated culture monitoring system of any one of the preceding claims, wherein the integrated culture monitoring system further comprises an output device displaying a graphical user interface (GUI) electronically coupled to the processor, and the processor is programmed to provide assay results to a system user.

16. The integrated culture monitoring system of claim 15, wherein the one or more assays comprise a PCR assay for detecting the presence of bacteria.

17. The integrated culture monitoring system of claim 16, wherein the PCR assay comprises broad-range 16S PCR.

18. A method for conducting an assay or assays in a device, the method comprising the steps of:
(a) providing the integrated culture monitoring system of any of the preceding claims;
(b) providing a culture fluid in the incubation chamber;
(c) using the processor causing:
(i) the temperature control device to control temperature in the incubation chamber;
(ii) the pump to pump a sample of culture fluid from the incubation chamber into the fluid-handling cartridge; and/or
(iii) the fluid-handling cartridge to conduct one or more assays on a sample of the culture fluid.

19. The method of claim 18, wherein the processor causes the pump to pump a sample of culture fluid from the incubation camber into the fluid-handling cartridge and the one or more assays are performed on the sample at a regular time interval.

20. The method of claim 19, wherein the regular time interval is from about every two minutes to about every thirty minutes.

21. The method of any one of claims 18 to 20, wherein the one or more assays comprise an assay for identifying a bacterial species.

22. The method of any one of claims 18 to 20, wherein the one or more assays comprise an assay for performing antimicrobial susceptibility testing.

23. The method of any one of claims 18 to 20, wherein the one or more assays comprise two assays, an assay for identifying bacterial species and an assay for performing antimicrobial susceptibility testing in the same device.

24. The method of claim 23, wherein the identification of a bacterial species and antimicrobial susceptibility testing are both completed within about eight hours, within about six hours, within about four hours, within about two hours, or within about one hour.

25. The method of any one of claims 18 to 24, wherein the one or more assays comprise a PCR assay to detect the presence or absence of bacteria in the sample and/or comprises a PCR assay for antimicrobial susceptibility testing.

26. A method for detecting and characterizing bacteria in a sample, the method comprising the steps of:
(a) providing an integrated culture monitoring system;
(b) obtaining a first sample from the incubation chamber and transporting the first sample through a fluid passage to a fluid-handling cartridge;
(c) performing sample preparation steps on the cartridge using droplet operations; and
(d) performing one or more assays on the prepared first sample to detect the presence or absence of bacteria and/or to characterize bacteria in the first sample on the fluid-handling cartridge.

27. The method for detecting and characterizing bacteria in a sample of claim 26, wherein the fluid-handling cartridge is a digital microfluidics (DMF) cartridge.

28. The method for detecting and characterizing bacteria in a sample of any one of claims 26 to 27, wherein the integrated culture monitoring system comprises the integrated culture monitoring system of any one of claims 1 to 17.

29. The method for detecting and characterizing bacteria in a sample of any one of claims 26 to 28, wherein the first sample of culture fluid is obtained from the incubation camber and transported into the fluid-handling cartridge and the one or more assays are performed on the sample at a regular time interval.

30. The method for detecting and characterizing bacteria in a sample of claim 29, wherein the regular time interval is from about every two minutes to about every thirty minutes.

31. The method for detecting and characterizing bacteria in a sample of any one of claims 26 to 30, wherein the one or more assays comprises an assay for identifying a bacterial species.

32. The method for detecting and characterizing bacteria in a sample of any one of claims 26 to 31, wherein the method further comprises the following steps if bacteria is detected in step (d):
(e) obtaining a second sample from the incubation chamber and transporting the second sample through the fluid passage to the fluid-handling cartridge;
(f) performing sample preparation steps on the fluid-handling cartridge using droplet operations; and
(g) performing an assay on the prepared second sample to determine bacterial species and to inform antibiotic susceptibility in the second sample on the fluid-handling cartridge.

33. The method for detecting and characterizing bacteria in a sample of claim 34, wherein the identification of a bacterial species and antimicrobial susceptibility testing are both completed within about eight hours, within about six hours, within about four hours, within about two hours, or within about one hour.

34. The method for detecting and characterizing bacteria in a sample of any one of claims 26 to 33, wherein the one or more assays comprises a PCR assay to detect the presence or absence of bacteria in the sample and/or comprises a PCR assay for antimicrobial susceptibility testing.

35. A method for detecting the presence or absence of bacteria in a sample, the method comprising the steps of:
(a) providing an integrated culture monitoring system, wherein the integrated monitoring system comprises an incubation chamber for holding a sample, a digital microfluidics (DMF) cartridge, a processor and a graphical user interface (GUI);
(b) providing a fluid connection between the incubation chamber and the DMF cartridge;
(c) causing a processor to:
(i) activate the integrated culture monitoring system to flush a liquid interface between the incubation chamber and the DMF cartridge; and/or
(ii) initiate a pump to pump a first sample or a first portion of the first sample from the incubation chamber into the DMF cartridge;
(d) performing an assay to detect the presence or absence of bacteria on the cartridge;
(e) processing the assay information using the processor; and
(f) reporting the assay results via the GUI of the system.

36. The method for detecting the presence or absence of bacteria in a sample of claim 35, wherein step (d) comprises performing broad-range 16S PCR on the first pumped sample or the first portion of the first pumped sample to detect the presence or absence of bacteria on the DMF cartridge.

37. The method of any one of claims 35 to 36, further comprising the step of transporting the first sample or the first portion of the first sample to a waste receptacle.

38. The method of any one of claims 35 to 37, wherein the method further comprises the following steps if bacteria are detected in the first pumped sample or in the first portion of the first pumped sample by an assay:
(a) using the processor to:
(i) activate the integrated culture monitoring system of any of the preceding claims to flush the liquid interface between the incubation chamber and the DMF cartridge; and
(ii) initiate the pump to obtain a second sample or a second portion of the first sample and transport the sample through the fluid passage to the DMF cartridge;
(b) performing sample preparation on the second pumped sample or the second portion of the first pumped sample on the DMF cartridge;
(c) performing a molecular identification (ID) test on the bacteria to determine a bacterial species;
(d) processing the test information using the processor; and
(e) reporting the test results via the GUI of the system.

39. The method of claim 38, wherein if the presence of bacteria is detected, the method further comprising:
using the processor to:
activate the integrated culture monitoring system of any of the preceding claims to flush the liquid interface between the incubation chamber and the DMF cartridge.

40. The method of any one of claims 35 to 39, wherein the method further comprises using the processor to cause the pump to pump a third sample, a second portion of the second sample, or a third portion of the first sample from the incubation chamber into the DMF cartridge.

41. The method of claim 40, wherein the processor causes the pump to pump a third sample, a second portion of the second sample, or a third portion of the first sample from the incubation chamber into the DMF cartridge at a regular time interval.

42. The method of claim 41, wherein the regular time interval is from about every two minutes to about every thirty minutes.

43. The method of any one of claims 40 to 42, wherein the method further comprises using the processor to cause the DMF cartridge to perform sample preparation on the third sample, on a second portion of the second sample, or on the third portion of the first sample.

44. The method of claim 43, wherein the method further comprises using the processor to cause the DMF cartridge to perform molecular and/or phenotypic antibiotic susceptibility testing (AST) on the third prepared sample, second portion of the second prepared sample, or the third portion of the first prepared sample to determine the presence or absence of antibiotic susceptibility gene markers.

45. The method of any one of claims 35 to 44, wherein the method further comprises determining Gram status of the detected bacteria.

46. The method of any one of claims 35 to 45, wherein the method further comprises using the processor to analyze the assay, the ID test, and/or the AST test results.

47. The method of claim 46, wherein the method further comprises using the processor to report the assay, the ID test, and/or the AST test results via the GUI of the system.

48. The method of any one of claims 35 to 47, wherein the DMF cartridge performs host response testing on a sample.

49. The method of claim 44, wherein the method further comprises performing a molecular ID test, molecular AST test and/or phenotypic AST test, and Gram status test concurrently on the third prepared sample, the second portion of the second prepared sample, or on the third portion of the first prepared sample.

50. The method of any one of claims 38 to 49, wherein the molecular ID test detects bacteria from a group consisting of E. coli, S. agalactiae, S. aureus, S. epidermidis, and Klebsiella pneumoniae.

51. The method of any one of claims 42 to 50, wherein the molecular AST/phenotypic AST test detects antibiotic gene markers from a group consisting of mec A/C, aminoglycosides, and carbapenem-resistant Enterobacteriaceae.

52. The method of any one of claims 43 to 51, wherein the Gram status test detects the presence of Gram-positive and/or Gram-negative bacteria.

53. The method of any one of claims 35 to 52, wherein the detection of bacteria, the Gram status test, ID test, and/or the AST test are completed within about eight hours, within about six hours, within about four hours, within about two hours, or within about one hour.

54. The method of any one of the preceding claims, wherein the incubation chamber is a liquid receptacle.

55. The method of claim 54, wherein the liquid receptacle is a culture bottle.

56. The method of any one of claims 35 to 53, wherein the molecular ID test, molecular AST test, phenotypic AST test, and/or Gram status test comprises performing PCR-based testing.

57. The integrated culture monitoring system of claim 3, wherein the reagents are selected from a group consisting of sample purification reagents, sample lysis reagents, and/or sample concentration reagents.

58. The integrated culture monitoring system of claim 3, wherein the reagents are selected from a group consisting of reagents required to perform broad-range 16S PCR, molecular ID PCR, molecular AST PCR, phenotypic AST PCR, and/or Gram status PCR.

59. The integrated culture monitoring system of any one of claims 57 to 58, wherein the reagents comprise dried reagents that are reconstituted prior to use.

60. The integrated culture monitoring system of any of the preceding claims, wherein the fluid-handling cartridge is disposable.

61. The integrated culture monitoring system of any of the preceding claims, wherein the sample is selected from a group consisting of a subject's blood sample, blood fraction, or other physiologically-derived samples such as swabs, urine, or cerebrospinal fluid.
